(19) United States
(12) Patent Application Publication
(10) Pub. No.: US 2022/0016286 A1
(43) Pub. Date: Jan. 20, 2022

(54) STERILIZATION STATUS DETECTION METHOD, STERILIZATION METHOD, STERILIZATION STATUS DETECTION DEVICE, STERILIZATION DEVICE

(71) Applicant: Ushio Denki Kabushiki Kaisha, Chiyoda-ku, Tokyo, (JP)

(72) Inventor: Keisuke Naito, Tokyo (JP)

(73) Assignee: Ushio Denki Kabushiki Kaisha, Chiyoda-ku, Tokyo, (JP)

(21) Appl. No.: 17/441,923

(22) PCT Filed: Mar. 27, 2020
(86) PCT No.: PCT/JP2020/013988

§ 371 Date: Sep. 22, 2021

(30) Foreign Application Priority Data
Mar. 29, 2019 (JP) 2019-066930

(51) Int. Cl. A61L 2/10; A61L 2/28

(52) U.S. Cl. CPC A61L 2/10; A61L 2/28

(57) ABSTRACT

A sterilization status detection method of the present invention includes:
a step (a) of irradiating an object with first ultraviolet light having a major emission wavelength of 200 nm or more and 230 nm or less;
a step (b) of detecting a decomposition product released from the object after the step (a); and
a step (c) of determining that sterilization treatment is necessary to the object when a detection intensity exceeds a threshold value, the detection intensity being an intensity of a detection signal in accordance with a detection concentration of the decomposition product detected at the step (b).

### TECHNICAL FIELD

The present invention relates to a sterilization status detection method and a sterilization status detection device that are capable of detecting whether sterilization status on an object has been completed. This present invention also relates to a sterilization method and a sterilization device having a function of the sterilization status detection.

### BACKGROUND ART

DNA exhibits the highest absorption characteristics around a wavelength of 260 nm. A low-pressure mercury lamp has a strong emission spectrum around a wavelength of 254 nm. Accordingly, a technique of using a low-pressure mercury lamp for sterilization has been widely available. (See Patent Document 1)

### CITATION LIST

### Patent Document

Patent Document 1: JP-A-2011-048968

### SUMMARY OF INVENTION

### Technical Problem

However, sterilization methods using ultraviolet light exhibit few changes that can be visually observed before and after the treatment, making it difficult to confirm the effectiveness of sterilization. Hence, even after the ultraviolet light has been irradiated to an object, it is not clear whether the sterilization has been completed to the object.

For example, if a large amount of bacteria adheres to the object, the bacteria will be stacked thickly on the surface of the object. When ultraviolet light is irradiated onto the surface of the object, the bacteria near the surface layer can be sterilized; however, the ultraviolet light does not reach the deeper layer of bacteria, in other words, the layer near the surface of the object, then the bacteria may not be reliably sterilized. In this way, when a large amount of bacteria adheres to an object, it may be more effective to sterilize the object using a method other than ultraviolet irradiation, such as cleaning with wiping.

In view of the above problem, it is an object of the present invention to provide a method for detecting a sterilization status for an object, and a sterilization status detection device to achieve the method. It is also an object of the present invention to provide a sterilization method and a sterilization device having a function of detecting the sterilization status.

### Solution to Problem

A sterilization status detection method of the present invention includes:

a step (a) of irradiating an object with first ultraviolet light having a major emission wavelength of 200 nm or more and 230 nm or less;

a step (b) of detecting a decomposition product released from the object after the step (a); and

a step (c) of determining that sterilization treatment is necessary to the object when a detection intensity exceeds a threshold value, the detection intensity being an intensity of a detection signal in accordance with a detection concentration of the decomposition product detected at the step (b).

As described above, DNA, which is contained in the nuclei of bacteria, exhibits the highest absorption characteristics at wavelengths near 260 nm. Hence, sterilization technology using low-pressure mercury lamps, which efficiently convert electricity into light and have a peak wavelength near 254 nm has been widely used.

Through intensive studies, the present inventor newly discovered a problem in which irradiating bacteria with ultraviolet light having a major emission wavelength (peak wavelength) of 200 nm or more and 230 nm or less (hereinafter refers to “first ultraviolet light”) generated an odor that had not been sensed before the irradiation. The present inventor infers that this odor is attributed to the following process that the first ultraviolet light is absorbed by protein contained in the bacteria, causing a portion of the molecular bonds of the protein to break and eventually form a new substance (decomposition product). In the course of the above research, the inventor also confirmed that when the bacteria were irradiated with ultraviolet light having a peak wavelength near 254 nm emitted from a low-pressure mercury lamp, there was almost no change in odor before and after the irradiation.

A larger amount of bacteria means a larger amount of protein contained in the bacteria, which leads to a larger amount of decomposition product. Hence, a high level of signal intensity (detection intensity) in accordance with the detection concentration of decomposition product indicates a large amount of bacteria. In contrast, in a case of the object to which the sterilization treatment has been completed, a small amount of decomposition product is produced by the irradiation of the first ultraviolet light because the object contains very few bacteria. Hence, in the case that the detection intensity exceeds a threshold value in the step (c), it is determined that the sterilization treatment has not been completed to the object and it is a status that the additional sterilization treatment is necessary.

Incidentally, ultraviolet light having a wavelength of 200 nm or more and 230 nm or less (first ultraviolet light), when irradiated to the skin of human bodies, is absorbed into the stratum corneum of the skin and does not penetrate further inward (the stratum basale side). Since stratum corneum cells contained in the stratum corneum are dead cells, the first ultraviolet light barely has a risk of damaging to DNA of the living cells, such as stratum spinosum, stratum granulosum, and dermis, unlike ultraviolet light having a wavelength of about 250 nm is irradiated to them and is absorbed to damage their DNA. Therefore, the first ultraviolet light has much less adverse effect on the human bodies than the ultraviolet light emitted from low-pressure mercury lamps.

The above method eliminates most of the need for considering a risk involved in the first ultraviolet light to human bodies even though the inspection is performed by irradiating the object with the first ultraviolet light at the time when human bodies happen to be present nearby. Moreover, this method enables the sterilization status to be detected by irradiating the object with the first ultraviolet light while an operator holds a sterilization detection device including a light source emitting the first ultraviolet light.

The above object can be any object as long as the sterilization treatment is necessary. Examples of the object are the one made from ceramics, metals, resins including plastics, rubbers including gaskets, wood, glass, and fibers such as cloth and paper.

The above decomposition product may be aldehydes. In this specification, the term “aldehydes” refers to compounds having at least one aldehyde group (also called “formyl group”) in their molecule. Examples of aldehydes are methyl propanal, methyl butanal, butanal, acetaldehyde, formaldehyde, propionaldehyde, pentanal, acrolein, and benzaldehyde.

In this case, the above step (b) may include a step of detecting aldehydes as the decomposition product with a sensor indicating a signal in accordance with the concentration of the aldehydes. Such sensors include high-sensitive tin oxide hot-wire sintered semiconductor sensors, ultra-sensitive zinc oxide substrate thin-film semiconductor sensors, quartz crystal microbalance (QCM) sensors, photoionization detectors (PID), Schottky diodes, metal oxide semiconductor (MOS) field-effect transistors, surface acoustic wave (SAW) devices, surface plasmon resonance sensors, or cantilevers.

The sterilization status detection method may include a step (d1) of detecting a substance of the same type as the decomposition product released from the object before the step (a), and a step (d2) of storing a reference intensity between the step (d1) and the step (a), the reference intensity being an intensity of a detection signal in accordance with the detection concentration of the decomposition product detected in the step (d1). In the step (c), the threshold value may be a value obtained by adding a predetermined tolerance to the reference intensity.

The above method allows an amount of a substance of the same type as decomposition product to be measured in advance and stored as a reference intensity, the substance being contained in the object itself or the ambient air and being different from the decomposition product generated from bacteria contained in the object by being irradiated with the first ultraviolet light. This procedure enables the threshold value as a comparison reference to be set, based on a consideration of the concentration of the decomposition product that is initially contained in the atmosphere in which the object is disposed. Hence, the sterilization status of the object in accordance with the amount of the decomposition product truly generated from bacteria is detected, enhancing the detection accuracy.

The step (b) may be a step of detecting the decomposition product after a predetermined time has elapsed after the step (a). The predetermined time may be set longer than a time required for the detection intensity to substantially match the reference intensity, the detection intensity being measured in the case that sterilization treatment has been completed to the object.

Even after the sterilization treatment is performed onto an object, it is difficult to expect a state in which there are no bacteria on the object from a socially accepted point of view. In other words, it is common to have a situation in which a certain amount of bacteria is present on the object even though additional sterilization treatment is not necessary. In such a situation, when the first ultraviolet light is irradiated to the object, it is absorbed with the bacteria (a small amount of bacteria) contained in the object to release the decomposition product, thus instantaneously increasing the detection concentration of the decomposition product. However, since the amount of the decomposition product is not very large, the detection concentration decreases with elapsed time, and eventually results in substantially matching the value of the detection concentration before the irradiation of the first ultraviolet light. In other words, the detection intensity substantially matches the reference intensity. It is noted that the term “substantially match” refers to a ratio of a difference value between the reference intensity and the detection intensity to the reference intensity is 5% or less.

Therefore, in the case that the amount of bacteria is small and no additional sterilization treatment is necessary, the signal intensity measured after the irradiation of the first ultraviolet light (detection intensity), which corresponds to the detection concentration of the decomposition product, substantially matches the signal intensity measured before the irradiation of the first ultraviolet light (reference intensity), which corresponds to the detection concentration of the decomposition product. This point will be described below with reference to FIG. 3.

In contrast, in the case that the object contains a large amount of bacteria, irradiating the object with the first ultraviolet light causes a large amount of bacteria contained in the object to release a large amount of the decomposition product, significantly increasing the concentration of the decomposition product contained in the environment near the object.

Hence, in the case of the object containing a large amount of bacteria, the detection intensity is initially high compared with that in the case of the object requiring no additional sterilization treatment. As a result, in the case of the object containing a large amount of bacteria, the detection intensity indicates a higher value than the reference intensity even after the predetermined time has elapsed since the irradiation of the first ultraviolet light.

The sterilization status detection method may include a step (e) of outputting a predetermined warning signal when the step (c) determines that the sterilization treatment is necessary to the object.

The warning signal may include sound signals, light signals, vibration signals and information signals that are transmitted to operation terminals, such as specific computers or smartphones, or servers.

A sterilization method of the present invention includes the sterilization status detection method described above and a step (f) of irradiating the object with the first ultraviolet light or a second ultraviolet light in a longer period than the step (a) when the sterilization treatment is determined to be necessary to the object in the step (c), the second ultraviolet light having a wavelength longer than that of the first ultraviolet light and a wavelength of 280 nm or less.

In the above method, the ultraviolet irradiation treatment for sterilization is performed only in the case that the additional sterilization treatment is determined to be necessary after detecting the sterilization status. This procedure substantially reduces the ultraviolet light irradiation time required for the sterilization, extending the service life of its light source.

A sterilization status detection device of the present invention includes:

a first light source that emits a first ultraviolet light having a major emission wavelength of 200 nm or more and 230 nm or less;

a sensor that outputs a detection signal by irradiating bacteria with the first ultraviolet light, the detection signal indicating an intensity corresponding to the concentration of decomposition product released from the bacteria; and

a determination unit that determines that sterilization treatment is necessary when a detection intensity exceeds a threshold value, the detection intensity being an intensity of the detection signal output from the sensor after the irradiation of the first ultraviolet light.

The above configuration is capable of readily determining, by irradiating an object with the first ultraviolet light, whether the sterilization treatment has been completed to the object and whether an additional sterilization treatment is necessary thereto.

The decomposition product may include aldehydes. The sensor may output the detection signal indicating an intensity corresponding to the contained concentration of the aldehydes.

The determination unit may determine that sterilization treatment is necessary when a difference between a reference intensity and the detection intensity exceeds a threshold value, the reference intensity being an intensity of the detection signal that is output from the sensor prior to the irradiation of the first ultraviolet light.

The detection intensity may be an intensity of the detection signal that is output from the sensor after a predetermined time has elapsed after the irradiation of the first ultraviolet light.

The sterilization status detection device may include a warning signal output unit that outputs a predetermined warning signal when the determination unit determines that sterilization treatment is necessary.

A sterilization device of the present invention includes:

the sterilization status detection device;

a second light source that emits a second ultraviolet light having a major emission wavelength longer than that of the first ultraviolet light and a wavelength of 200 nm or more and 280 nm or less; and

a light source control unit;

wherein the light source control unit controls the second light source to emit the second ultraviolet light when the determination unit determines that sterilization treatment is necessary.

A sterilization device of the present invention includes:

the sterilization status detection device; and

a light source control unit;

wherein the light source control unit controls the first light source to emit the first ultraviolet light at a longer period than the time of emitting the first ultraviolet light for the sensor to detect the decomposition product, when the determination unit determines that the sterilization treatment is necessary.

### Advantageous Effects of Invention

According to the present invention, the sterilization status of an object is detected in a simple method. Also, according to the present invention, the sterilization treatment is performed only when an additional sterilization treatment is necessary.

### BRIEF DESCRIPTION OF DRAWINGS

FIG. 1 is a block diagram schematically illustrating a configuration of an embodiment of a sterilization status detection device of the present invention.

FIG. 2 is a flow chart describing a procedure of a sterilization status detection method of the present invention.

FIG. 3 is a graph illustrating a variation of the intensity Yi of the detection concentration signal (detection signal) of a decomposition product after the irradiation of the first ultraviolet light.

FIG. 4 is a flow chart describing another procedure of a sterilization status detection method of the present invention.

FIG. 5 is a block diagram schematically illustrating a configuration of another embodiment of a sterilization device of the present invention.

FIG. 6 is a flow chart describing a procedure of a sterilization method of the present invention.

FIG. 7 is a block diagram schematically illustrating a configuration of an embodiment of a sterilization device of the present invention.

FIG. 8 is a flow chart describing another procedure of a sterilization method of the present invention.

FIG. 9 is a graph illustrating a variation of the intensity Yi of the detection concentration signal (detection signal) of a decomposition product to explain another procedure of a sterilization status detection method of the present invention.

### DESCRIPTION OF EMBODIMENTS

Hereinafter, embodiments of a sterilization status detection method, a sterilization method, a sterilization status detection device and a sterilization device each will be described with reference to the drawings as appropriate.

### First Embodiment

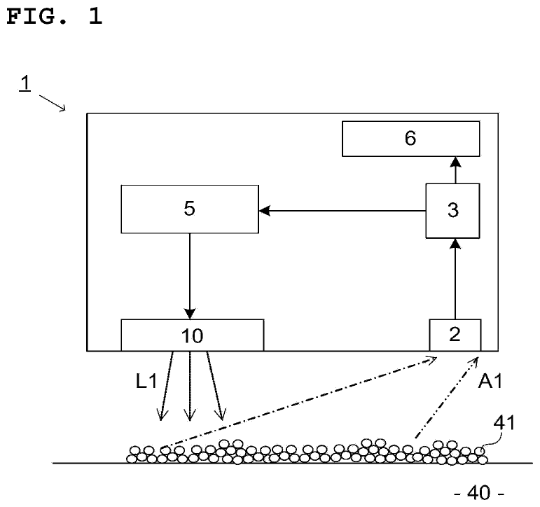

FIG. 1 is a block diagram schematically illustrating a configuration of an embodiment of a sterilization status detection device of the present invention. A sterilization status detection device 1 is provided with a first light source 10, a sensor 2, a determination unit 3, a light source control unit 5 and a warning signal output unit 6. FIG. 1 schematically illustrates a case in which a large amount of bacteria 41 is present on a surface of an object 40 for which the sterilization status is to be detected.

The first light source 10 is a light source capable of emitting a first ultraviolet light L1 having a major emission wavelength of 200 nm or more and 230 nm or less. For example, the first light source 10, when configured as an excimer lamp with KrCl as light-emitting gases, emits the first ultraviolet light L1 having a major emission wavelength of 222 nm. It is noted that the first light source 10 can be any configuration as long as it is a light source capable of emitting the first ultraviolet light having a major wavelength of 200 nm or more and 230 nm or less. The first light source 10 may be an excimer lamp with other materials including KrBr as light-emitting gases or a solid-state light source element including an LED and a laser diode. Furthermore, the first light source 10 may include a filter that cuts off light components having a wavelength of over 230 nm.

The term “major emission wavelength” refers to the wavelength λi such that the integrated intensity of a wavelength region Z(λi) constitutes 40% or more of the total integrated intensity within the emission spectrum, the wavelength region Z(λi) being defined as a wavelength region within the range of ±10 nm. In the case of a light source that has an extremely narrow half-width and a light intensity at only the specific wavelength, such as an excimer lamp, the wavelength having the highest intensity (major peak wavelength) may be normally considered to be a major emission wavelength.

The sensor 2 detects a decomposition product A1 that is produced by decomposing bacteria 41 to which the first ultraviolet light L1 is irradiated, and outputs a signal of an intensity (detection intensity) corresponding to the detection concentration of the decomposition product A1. The sensor 2 is, for example, an aldehyde sensor that detects the aldehydes as the decomposition product A1. Furthermore, the sensor 2 specifically includes high-sensitive tin oxide hot-wire sintered semiconductor sensors, ultra-sensitive zinc oxide substrate thin-film semiconductor sensors, quartz crystal microbalance (QCM) sensors, photoionization detectors (PID), Schottky diodes, metal oxide semiconductor (MOS) field-effect transistors, surface acoustic wave (SAW) devices, surface plasmon resonance sensors, or cantilevers.

The determination unit 3 is a means of arithmetic processing to determine whether or not an additional sterilization treatment is necessary to the object 40, and configured to be composed of software or a dedicated hardware.

The light source control unit 5 is a means of performing an emission control by performing an electric control to the first light source 10, and configured to include an electric circuit not shown.

The warning signal output unit 6 is, when the determination unit 3 determines that the additional sterilization treatment is necessary to the object 40, a means of outputting a warning signal to report the information. The warning signal can be, for example, audible signals, light signals, vibration signals or information signals. The warning signal output unit 6 represents embodiments according to the types of the warning signals; the warning signal output unit 6 can be, for example, speakers, visible light sources such as visible LEDs, vibrators or information transmitter units. In the case of the warning signal being the information signal, the information signal describing the information that “The additional sterilization treatment is necessary to the object 40” may be transmitted to portable devices including specific computers or smartphones, or servers via wired cable or wireless communication.

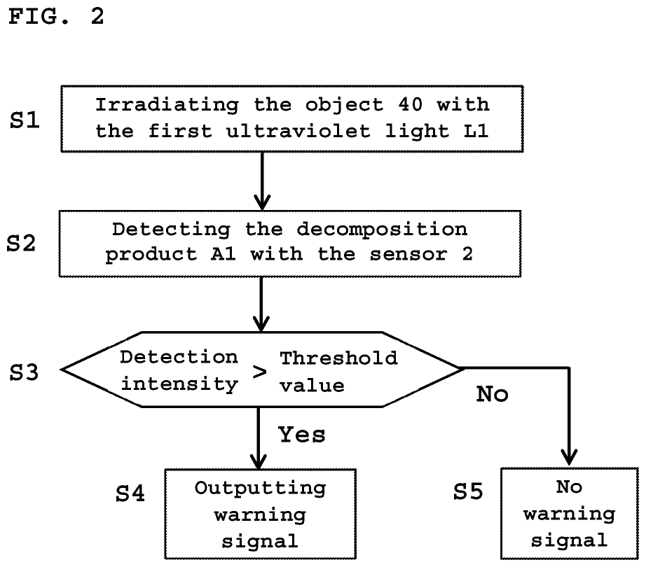

A method of detecting a sterilization status on the object 40 by the sterilization status detection device 1 is described with reference to the flow chart shown in FIG. 2. The signs of the following steps each correspond to the steps in FIG. 2.

### (Step S1)

The light source control unit 5 controls the first light source 10 to irradiate the object 40 with the first ultraviolet light L1. The sterilization status detection device 1 may be provided with a window (not shown) to allow the first ultraviolet light L1 emitted from the first light source 10 to be irradiated outside the sterilization status detection device 1.

In the step S1, the light source control unit 5 appropriately sets the emission time of the first light source 10. The emission time ranges, for example, from 1 second to 3 minutes, more preferably, from 10 seconds to 2 minutes.

The step S1 corresponds to the step (a).

### (Step S2)

The sensor 2 detects the decomposition product A1 released from the object 40 and contained in the atmosphere. As described above, when the sensor 2 is, for example, an aldehyde sensor, the sensor outputs a detection signal to the determination unit 3, the detection signal representing an intensity corresponding to the detection concentration of the aldehydes as the decomposition product A1.

The step S2 corresponds to the step (b).

The determination unit 3 compares the intensity of the detection signal (detection intensity) with a threshold value. The determination unit 3 may store the information related to the threshold value as a comparative reference in advance.

When the detection intensity exceeds the threshold value (Yes in the step S3), the determination unit 3 determines that an additional sterilization treatment is necessary to the object 40 because a large amount of the decomposition product A1 released from the object 40 indicates a large amount of the bacteria 41 contained therein. In the present embodiment, the warning signal output unit 6 outputs the warning signal based on the instruction signal from the determination unit 3 (step S4). Examples of the warning signals are, as described above, audible signals, light signals, vibration signals or information signals. In the case that the detection intensity is not more than the threshold value (No in the step S3), the warning signal output unit 6 does not output the warning signal (step S5).

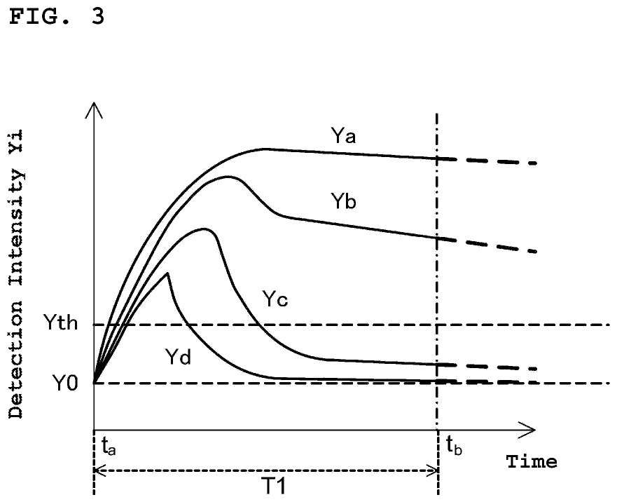

FIG. 3 is a graph illustrating a variation of the detection intensity Yi of the decomposition product A1 over time after the first ultraviolet light L1 has been irradiated to four types of the objects 40 that contain the different amount of bacteria 41 (Ya, Yb, Yc, Yd). In FIG. 3, the horizontal axis corresponds to the time, and the vertical axis corresponds to the detection intensity Yi. FIG. 3 illustrates a variation of the detection intensity Yi over time after the time to when the first ultraviolet light L1 is irradiated to the object 40.

In the case that the amount of the bacteria 41 is relatively small (Yc, Yd), irradiating the object 40 with the first ultraviolet light L1 allows the detection intensity Yi to temporarily increase at first and turn to decrease with time elapsed. This variation of the detection intensity is explained by the following reason that since the amount of the decomposition product A1 contained in the atmosphere, in other words, the concentration of the decomposition product A1 is small, it spreads with time elapsed, thereby decreasing the concentration of the decomposition product A1 detected with the sensor 2.

In contrast, in the case that the amount of the bacteria 41 is relatively large (Ya, Yb), the detection intensity Yi remains a high value even after a certain amount of time T1 has elapsed. This is explained by the reason that the concentration of the decomposition product A1 contained in the atmosphere is high. For this reason, the detection intensity Yi is still high even after the time T1 has elapsed, compared with the case in which the amount of the bacteria 41 is relatively small (Yc, Yd).

Hence, the determination unit 3 compares the detection intensity Yi with the threshold value Yth after the predetermined time T1 has elapsed. In the case that the detection intensity Yi exceeds the threshold value Yth, which indicates a large amount of bacteria 41 being contained in the object 40, the determination unit 3 determines that an additional sterilization treatment is necessary.

The step S3 corresponds to the step (c) and the step S4 corresponds to the step (e).

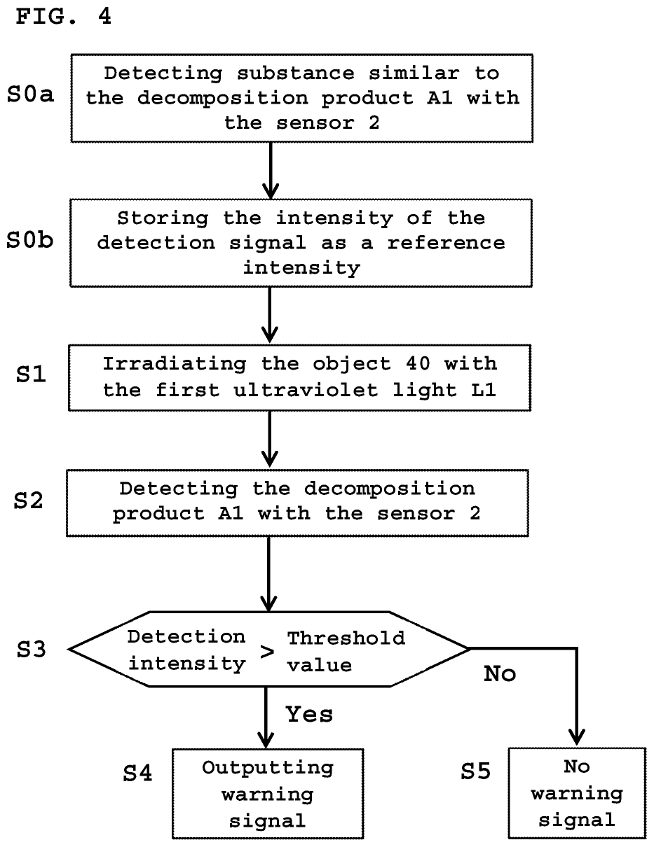

The sensor 2 may detect a substance of the same type as the decomposition product A1 and measure a signal corresponding to the detection concentration (reference signal) prior to the performance of the step S1, in other words, before irradiating with the first ultraviolet light L1. (See the FIG. 4) FIG. 4 is a flow chart that describes another procedure of the sterilization status detection method according to the present invention.

(step S0a, step S0b)

The sensor 2 detects a substance of the same type as the decomposition product A1 contained in the atmosphere at a stage prior to the step S1 of irradiating with the first ultraviolet light L1 (step S0a). The sensor 2 outputs a signal (reference signal) to the determination unit 3, the signal indicating an intensity corresponding to the detection concentration of the substance of the same type as the decomposition product A1. The determination unit 3 stores the intensity of the reference signal as the reference intensity Y0. (step S0b)

The step S0a corresponds to the step (d1) and the step S0b corresponds to the step (d2).

In this way, in the case that the determination unit 3 stores the reference intensity Y0, the threshold value Yth, which is a comparative reference on the step S3, may be set based on the reference intensity Y0. The threshold value Yth can be set, for example, as a value that an acceptable predetermined value (tolerance) is added to the reference intensity Y0. An example of the threshold value Yth can be a value of 150% of the reference intensity Y0.

The sterilization status detection device 1 readily informs that the sterilization treatment is not sufficient to the object 40 and the additional sterilization treatment is necessary when the warning signal output unit 6 outputs the warning signal.

As described above with reference to the FIG. 3, in the case of relatively small amount of bacteria 41 (Yc, Yd), irradiating the object 40 with the first ultraviolet light L1 allows the detection intensity Yi to temporarily increase at first, turn to decrease with time elapsed and become nearly the same value as the one before the irradiation. In the example of the FIG. 3, the status Yd indicates that the detection intensity Yi has become nearly the same detection concentration Yi with the time T1 elapsed after irradiating with the first ultraviolet light L1.

In contrast, in the case of relatively large amount of bacteria 41 (Ya, Yb), irradiating the object 40 with the first ultraviolet light L1 allows the detection intensity Yi to significantly increase at first, and turn to moderately decrease with time elapsed. However, it takes considerable time for the detection intensity Yi to become nearly the same value as the one before the irradiation.

Hence, a predetermined time is, for example, set to be the time T1, and the comparison between the detection intensity and the threshold value (step S3) may be performed after the predetermined time (time T1) has elapsed after irradiating with the first ultraviolet light L1. In the case of the small amount of bacteria 41 contained in the object 40, the measured detection concentration Yi indicates the same value as the detection concentration before irradiating with the first ultraviolet light L1 (reference intensity Y0), thereby determining that the status is that no additional sterilization is necessary and the sterilization has been completed. In this case the detection concentration Yi is obviously less than the threshold value Yth.

In contrast, in the case of the large amount of bacteria contained in the object 40, since the detection concentration Yi remains a high value (higher than the threshold value Yth) even after the predetermined time (the time T1 on the above example) has elapsed after irradiating with the first ultraviolet light L1, thereby determining that the status is that an additional sterilization is necessary.

### Second Embodiment

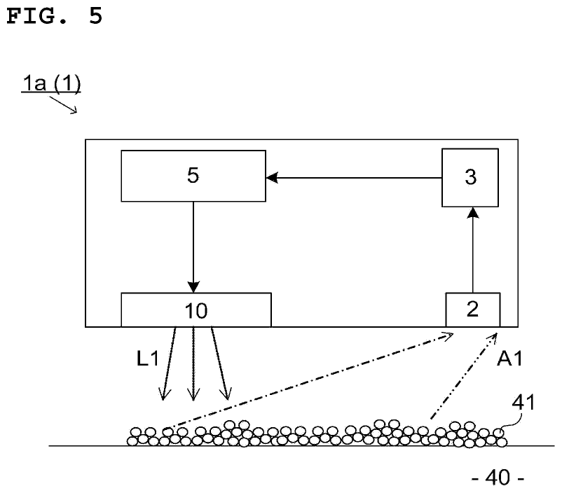

FIG. 5 is a block diagram schematically illustrating a configuration of an embodiment of the sterilization device according to the present invention. The elements that are common to those in the first embodiment are denoted with the identical signs, and their descriptions are omitted as appropriate.

A sterilization device 1a of the present embodiment is provided with the first light source 10, the sensor 2, the determination unit 3 and the light source control unit 5. FIG. 5, as similar to the FIG. 1, illustrates a case in which a large amount of the bacteria 41 is present on the surface of the object 40 to which the sterilization status is to be detected.

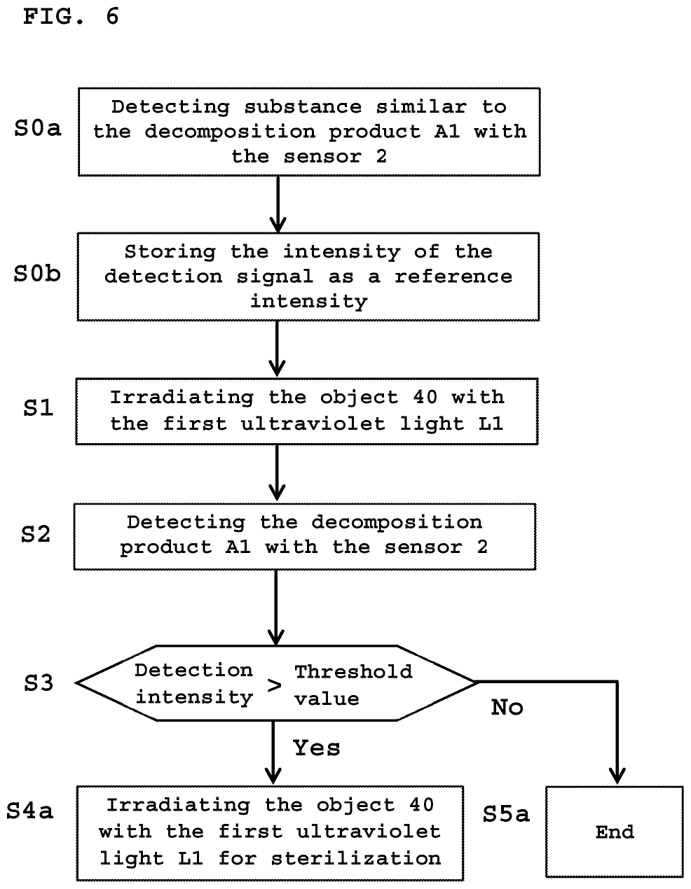

A method of sterilizing the object 40 with the sterilization device 1a is described with reference to a flow chart indicated in the FIG. 6. Note that the steps that are common to those in the FIG. 2 are denoted with the common step signs.

The step S1 to S3 are performed in a similar method of the first embodiment. As indicated in FIG. 6, a step S0a or a step S0b may be performed prior to the step S1.

(step S4a, step S5a)

In the first embodiment, in the case that the determination unit 3 determines that the detection intensity exceeds the threshold value (Yes in the step S3), the warning signal output unit 6 outputs the warning signal (step S4). In the present embodiment, the step S4 is replaced with a step S4a in which the light source control unit 5 controls the first light source 10 to irradiate the object 40 with the first ultraviolet light L1 for sterilization. The step S4a corresponds to the step (f).

The first ultraviolet light L1, which is emitted from the first light source 10, has a major emission wavelength of 200 nm or more and 230 nm or less. The emission wavelength is capable of sterilizing the bacteria 41 although it is slightly apart from the peak wavelength of the absorption spectrum of DNA. However, a larger amount of light intensity is required to be irradiated to sterilize the bacteria 41, compared with the amount of light intensity required to break the proteins bonds contained in the bacteria 41 to generate the decomposition product A1 in the step S1 for the detection of the sterilization status. Hence, the first ultraviolet light L1 with longer period of time and/or having a higher light intensity is irradiated to the object 40 in the step S4a than that in the step S1.

In contrast, in the case that the detection intensity is equal to or less than the threshold value (No in the step S3), the treatment has been completed without irradiating with the first ultraviolet light L1 from the first light source 10 (step S5a).

In the sterilization device 1a of the present embodiment, the step S3 determines whether the sterilization treatment is necessary to the object 40 in advance, and after that, only in the case of being determined necessary, the first ultraviolet light L1 is irradiated to the object 40 with the amount of irradiation required for sterilization. On the other hand, as described above, the amount of the first ultraviolet light L1 irradiated to detect the sterilization status of the object 40 in the step S1 is much less than the amount of the first ultraviolet light L1 irradiated for sterilization in the step S5a. As a result, the first light source 10 extends its service life compared with the case in which the first ultraviolet light L1 is irradiated for sterilization purposes even though the sterilization has already been completed.

The sterilization device 1a may also be provided with the warning signal output unit 6, as is similar to the sterilization status detection device 1 shown in FIG. 1. In this case, the sterilization device 1a can also be used as the sterilization status detection device 1 of the first embodiment.

For example, in the case in which a large amount of the bacteria 41 is stacked on the object 40, it may be difficult for ultraviolet light to be irradiated to the region in which the bacteria 41 overlap. In this case, it may be effective to perform the treatment of wiping the surface of the object 40 with, for example, alcohol. Under these circumstances, the determination unit 3 may determine whether the sterilization by the ultraviolet light L1 is performed or the warning signal is output according to the magnitude of the detection intensity detected by the sensor 2.

### Third Embodiment

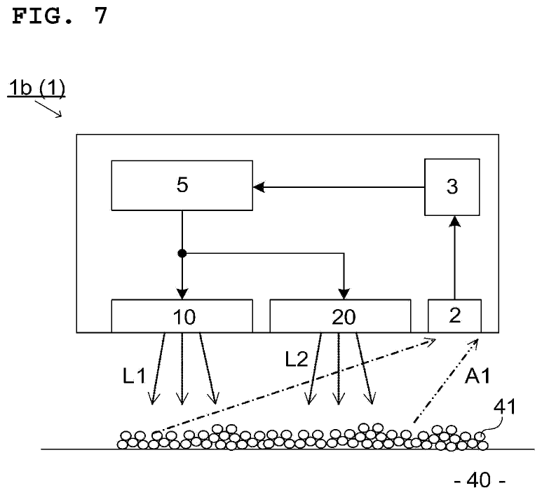

FIG. 7 is a block diagram schematically illustrating a configuration of an embodiment of a sterilization device of the present invention. The elements that are common to those in the first embodiment are denoted with the identical signs, and their descriptions are omitted as appropriate.

The sterilization device 1b of the present embodiment is further provided with a second light source 20, compared with the sterilization device 1a of the second embodiment.

The second light source 20 is a light source capable of emitting a second ultraviolet light L2 having a major emission wavelength longer than the first ultraviolet light L1, the major emission wavelength being 200 nm or more and 280 nm or less. The second light source 20 is appropriately configured, for example, to be a low-pressure mercury lamp from the viewpoint of excellent efficiency in electrical-to-optical conversion. The second light source 20 may also be a solid-state light source such as an LED or a laser diode.

The second light source 20, together with the first light source 10, is controlled by the light source control unit 5.

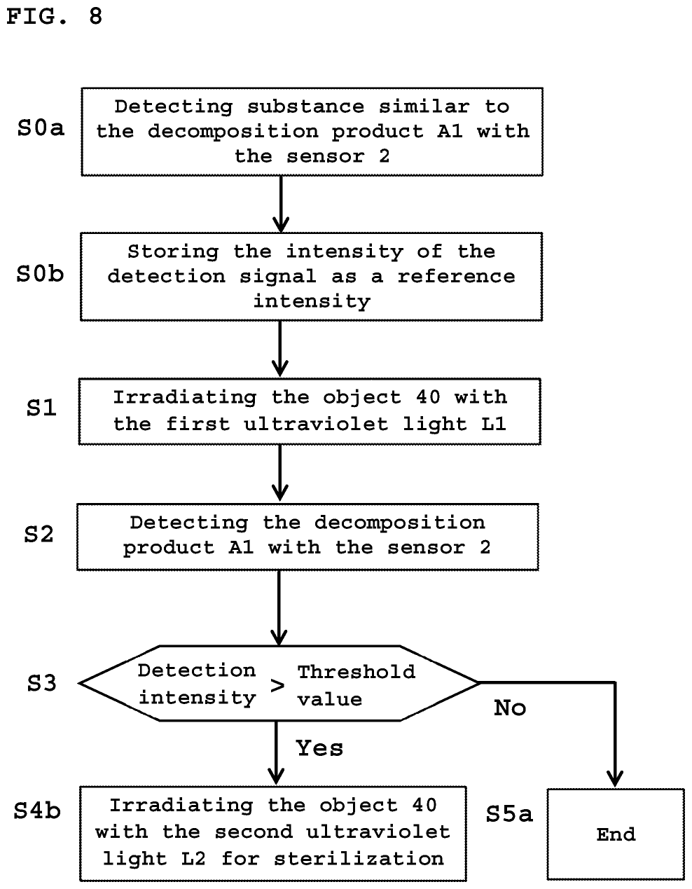

A method of sterilizing the object 40 with the sterilization device 1b is described with reference to a flow chart shown in FIG. 8. Note that the steps that are common to those in the FIG. 6 are denoted with the common step signs.

The flowchart shown in FIG. 8 only differs from the flowchart shown in FIG. 6 in that the step S4b is performed instead of the step S4a.

### (Step S4b)

In the second embodiment, in the case that the determination unit 3 determines that the detection intensity exceeds the threshold value (Yes in the step S3), the light source control unit 5 controls the first light source 10 to irradiate the object 40 with the first ultraviolet light L1 for sterilization (step S4a). In contrast, in the present embodiment, in the case that the determination unit 3 determines that the detection intensity exceeds the threshold value, the light source control unit 5 controls the second light source 20 to irradiate the object 40 with the second ultraviolet light L2 for sterilization (step S4b). This step S4b corresponds to the step (f).

The second ultraviolet light L2 emitted from the second ultraviolet light source 20 has a major emission wavelength closer to the peak wavelength of the absorption spectrum of DNA than that of the first ultraviolet light L1. In particular, the second light source, when constituted by a low-pressure mercury lamp 20, has a higher efficiency in electrical-to-optical conversion compared with the first light source 10, and is available at a lower cost.

In the present embodiment, as is similar to the second embodiment, the step S3 determines whether the sterilization treatment is necessary to the object 40 in advance, and after that, only in the case of being determined necessary, the ultraviolet light for sterilization (in this case, the second ultraviolet light L2) is irradiated to the object 40. The second light source 20 is capable of performing sterilization more efficiently than the first light source 10, thus the second light source 20 additionally provided for sterilization enables the first light source 10 to extend its service life.

The sterilization device 1b may also be provided with the warning signal output unit 6, as is similar to the sterilization status detection device 1 shown in FIG. 1. In this case, the sterilization device 1b can also be used as the sterilization status detection device 1 of the first embodiment.

For example, in the case in which a large amount of the bacteria 41 is stacked on the object 40, it may be difficult for ultraviolet light to be irradiated to the region in which the bacteria 41 overlap. In this case, it may be effective to perform the treatment of wiping the surface of the object with, for example, alcohol. The determination unit 3 may determine whether the sterilization by the ultraviolet light L2 is performed or the warning signal is output according to the magnitude of the detection intensity detected by the sensor 2.

### Another Embodiment

Hereinafter, another embodiment is described.

<1> In the above first embodiment, a case is described using the sterilization status detection device 1, the case in which the sterilization status of the object 40 being detected by performing each step of the steps S1 to S5. However, the sterilization status detection device 1 may not be used as long as the steps S1 to S5 are performed. In other words, the first light source 10 that emits the first ultraviolet light L1, the sensor 2 that detects the decomposition product A1, the determination unit 3 that determines the sterilization status of the object 40 based on the detection signal from the sensor 2, and the warning signal output unit 6 are not always mounted in the same device.

<2> The sterilization status detection device 1 of the first embodiment may be provided with a motion sensor that is not shown. The light source control unit 5 controls the first light source 10 to regularly emit the light in a predetermined time interval to perform the above steps S1 to S5; however, in the case that the motion sensor detects humans being present nearby, the light source control 5 may control the first light source 10 to stop emitting the light therefrom.

As described above, the first ultraviolet light L1 having a major emission wavelength of 200 nm or more and 230 nm or less has little adverse effect on human bodies; however, there are more than a few people who feel uncomfortable with the ultraviolet light being exposed thereto. In the above configuration, the irradiation of the first ultraviolet light L1 automatically stops when humans are present nearby, thus making it easier to introduce the sterilization status detection device 1 in places where people come and go.

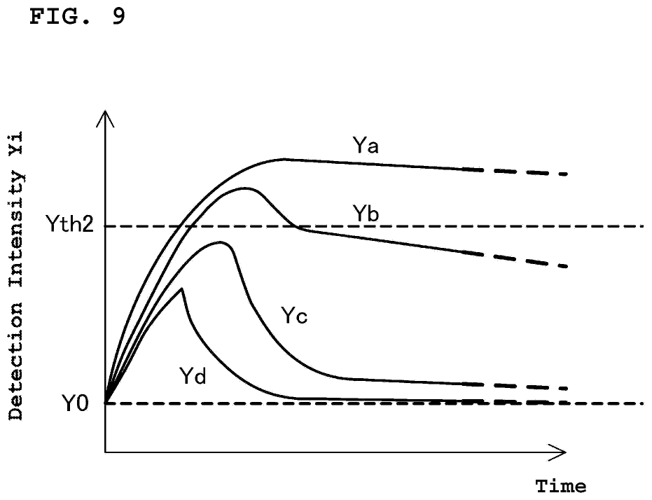

The same configuration may also be applied to the sterilization device (1a, 1b).

<3> In the above embodiment, whether or not an additional sterilization to the object 40 being necessary is determined by comparing the threshold value Yth with the detection intensity Yi of the sensor 2 after the predetermined time has elapsed after irradiating the object 40 with the first ultraviolet light L1 in the step S1. However, as described above, in the case that the object 40 contains a large amount of bacteria 41 (status Ya, Yb in the FIG. 3), the value of the detection intensity Yi significantly increases even just after the irradiation of the first ultraviolet light L1. Hence, as shown in FIG. 9, in the case in which the detection intensity Yi exceeds a predetermined value Yth2 regardless of after the irradiation of the ultraviolet light L1 or during the irradiation thereof, an additional sterilization may be determined to be necessary. In other words, the steps S2 to S3 may be performed during the irradiation of the first ultraviolet light L1.

It is noted that the threshold value Yth2, which is used in the case of determining the necessity of the sterilization in this method, is set to be a higher value than the threshold value Yth, which is used in the method described in the above embodiment.

### REFERENCE SIGNS LIST

1 sterilization status detection device
1a, 1b sterilization device
2 sensor
3 determination unit
5 light source control unit
6 warning signal output unit
10 first light source
20 second light source
40 object
41 bacteria
A1 decomposition product
L1 first ultraviolet light
L2 second ultraviolet light

1. A sterilization status detection method comprising:
a step (a) of irradiating an object with first ultraviolet light having a major emission wavelength of 200 nm or more and 230 nm or less;
a step (b) of detecting a decomposition product released from the object after the step (a); and
a step (c) of determining that sterilization treatment is necessary to the object when a detection intensity exceeds a threshold value, the detection intensity being an intensity of a detection signal in accordance with a detection concentration of the decomposition product detected at the step (b).

2. The sterilization status detection method according to claim 1, wherein the decomposition product is aldehydes.

3. The sterilization status detection method according to claim 2, wherein the step (b) includes a step of detecting aldehydes as the decomposition product with a sensor indicating a signal in accordance with the concentration of the aldehydes.

4. The sterilization status detection method according to claim 1, further comprising a step (d1) of detecting a substance of the same type as the decomposition product released from the object before the step (a); and
a step (d2) of storing a reference intensity between the step (d1) and the step (a), the reference intensity being an intensity of a detection signal in accordance with the detection concentration of the decomposition product detected in the step (d1);
wherein the threshold value is a value obtained by adding a predetermined tolerance to the reference intensity in the step (c).

5. The sterilization status detection method according to claim 4, wherein the step (b) is a step of detecting the decomposition product after a predetermined time has elapsed after the step (a) and,
the predetermined time is set longer than a time required for the detection intensity to substantially match the reference intensity, the detection intensity being measured in the case that sterilization treatment has been completed to the object.

6. The sterilization status detection method according to claim 1, further comprising a step (e) of outputting a predetermined warning signal when the step (c) determines that the sterilization treatment is necessary to the object.

7. A sterilization method comprising:
the sterilization status detection method according to claim 1, and
a step (f) of irradiating the object with the first ultraviolet light or a second ultraviolet light in a longer period than the step (a) when the sterilization treatment is determined to be necessary to the object in the step (c), the second ultraviolet light having a wavelength longer than that of the first ultraviolet light and a wavelength of 280 nm or less.

8. A sterilization status detection device comprising:
a first light source that emits a first ultraviolet light having a major emission wavelength of 200 nm or more and 230 nm or less;
a sensor that outputs a detection signal by irradiating bacteria with the first ultraviolet light, the detection signal indicating an intensity corresponding to the concentration of decomposition product released from the bacteria; and
a determination unit that determines that sterilization treatment is necessary when a detection intensity exceeds a threshold value, the detection intensity being an intensity of the detection signal output from the sensor after the irradiation of the first ultraviolet light.

9. The sterilization status detection device according to claim 8, wherein the decomposition product is aldehydes, and the sensor outputs the detection signal indicating an intensity corresponding to the contained concentration of the aldehydes.

10. The sterilization status detection device according to claim 8, wherein the determination unit determines that sterilization treatment is necessary when a difference between a reference intensity and the detection intensity exceeds a threshold value, the reference intensity being an intensity of the detection signal that is output from the sensor prior to the irradiation of the first ultraviolet light.

11. The sterilization status detection device according to claim 8, wherein the detection intensity is an intensity of the detection signal that is output from the sensor after a predetermined time has elapsed after the irradiation of the first ultraviolet light.

12. The sterilization status detection device according to claim 8, further comprising a warning signal output unit that outputs a predetermined warning signal when the determination unit determines that the sterilization treatment is necessary.

13. A sterilization device comprising:
the sterilization status detection device according to claim 8;
a second light source that emits a second ultraviolet light having a major emission wavelength longer than that of the first ultraviolet light and a wavelength of 200 nm or more and 280 nm or less; and
a light source control unit;
wherein the light source control unit controls the second light source to emit the second ultraviolet light when the determination unit determines that sterilization treatment is necessary.

14. A sterilization device comprising:
the sterilization status detection device according to claim 8; and
a light source control unit;
wherein the light source control unit controls the first light source to emit the first ultraviolet light at a longer period than the time of emitting the first ultraviolet light for the sensor to detect the decomposition product, when the determination unit determines that the sterilization treatment is necessary.

15. The sterilization status detection method according to claim 2, further comprising a step (d1) of detecting a substance of the same type as the decomposition product released from the object before the step (a); and
a step (d2) of storing a reference intensity between the step (d1) and the step (a), the reference intensity being an intensity of a detection signal in accordance with the detection concentration of the decomposition product detected in the step (d1);
wherein the threshold value is a value obtained by adding a predetermined tolerance to the reference intensity in the step (c).

16. The sterilization status detection method according to claim 3, further comprising a step (d1) of detecting a substance of the same type as the decomposition product released from the object before the step (a); and
a step (d2) of storing a reference intensity between the step (d1) and the step (a), the reference intensity being an intensity of a detection signal in accordance with the detection concentration of the decomposition product detected in the step (d1);
wherein the threshold value is a value obtained by adding a predetermined tolerance to the reference intensity in the step (c).

17. The sterilization status detection method according to claim 2, further comprising a step (e) of outputting a predetermined warning signal when the step (c) determines that the sterilization treatment is necessary to the object.

18. The sterilization status detection device according to claim 9, wherein the determination unit determines that sterilization treatment is necessary when a difference between a reference intensity and the detection intensity exceeds a threshold value, the reference intensity being an intensity of the detection signal that is output from the sensor prior to the irradiation of the first ultraviolet light.

19. A sterilization device comprising:
the sterilization status detection device according to claim 9;
a second light source that emits a second ultraviolet light having a major emission wavelength longer than that of the first ultraviolet light and a wavelength of 200 nm or more and 280 nm or less; and
a light source control unit;
wherein the light source control unit controls the second light source to emit the second ultraviolet light when the determination unit determines that sterilization treatment is necessary.

20. A sterilization device comprising:
the sterilization status detection device according to claim 9; and
a light source control unit;
wherein the light source control unit controls the first light source to emit the first ultraviolet light at a longer period than the time of emitting the first ultraviolet light for the sensor to detect the decomposition product, when the determination unit determines that the sterilization treatment is necessary.
